(19) United States
(12) Patent Application Publication
(10) Pub. No.: US 2026/0325588 A1
(43) Pub. Date: Oct. 22, 2026

(54) SYSTEM AND METHOD FOR AFFECTING INTESTINAL MICROBIAL FLORA

(71) Applicant: MetaModix, Inc., Plymouth, MN (US)

(72) Inventors: Kedar R. Belhe, Minnetonka, MN (US); Todd Stangenes, Minneapolis, MN (US)

(21) Appl. No.: 19/633,130

(22) Filed: Mar. 30, 2026

Related U.S. Application Data
(63) Continuation of application No. 18/371,672, filed on Sep. 22, 2023, now abandoned.

(51) Int. Cl. A61K 35/741; A61F 2/04; A61F 5/00

(52) U.S. Cl. CPC A61K 35/741; A61F 2/04; A61F 5/0076; A61F 2002/044; A61F 2002/045

(57) ABSTRACT

Described is a system for affecting intestinal microbial flora. The system includes an intestinal sleeve that is implanted in a patient and bypasses a section of the intestine; and probiotics, prebiotics or pharmacologic therapy used in combination with the intestinal sleeve. Also described are related methods.

### CROSS-REFERENCE TO RELATED APPLICATIONS

This application is a continuation of U.S. application Ser. No. 14/156,209 filed Jan. 15, 2014 which claims priority to Provisional Application No. 61/752,839, filed Jan. 15, 2013, which is herein incorporated by reference in its entirety. This application is related to the following commonly assigned applications and patents, each of which is incorporated herein by reference: (1) U.S. Pat. No. 8,211,186; (2) U.S. Pat. No. 8,282,598; (3) U.S. Patent Application Publication 2011/0106273, published May 5, 2011; (4) U.S. Patent Application Publication 2012/0065571, published Mar. 15, 2012; (5) U.S. Patent Application Publication 2012/0184893, published Jul. 19, 2012; (6) U.S. Patent Application Publication 2013/0030351, published Jan. 31, 2013; and (7) U.S. Patent Application Publication 2012/0302936, published Nov. 29, 2012.

### TECHNICAL FIELD

This invention relates to implants that are placed within gastrointestinal organs including in the distal portion of the stomach and the small intestine. In particular, it relates to an implant system and method that can be placed by endoscopic means, in order to reduce colonization of the intestines by certain bacteria and treat chronic diseases.

### BACKGROUND

Recent clinical evidence suggests that surgical treatments for obesity, such as Rouen-Y gastric bypass (RYGB) can cause remission of diabetes in 75%-80% of patients. Hence, minimally invasive procedures including intra-luminal gastrointestinal bypass implants have recently been proposed to mimic all or some of the anatomical, physiological and metabolic changes achieved by a Rouen-Y gastric bypass. Some of these procedures provide a means to internally bypass a portion of the small intestine. It has been observed through anecdotal clinical evidence that the effect of such bypass means may be causing remission of metabolic disorders, which persists months after the bypass means is removed from a patient.

Current pharmaceutical approaches to modifying intestinal microbial flora involve the administration of therapy to selectively ameliorate “bad” (having a negative effect on the health of a patient) bacteria in the intestines in order to promote colonization by “good” (having a positive effect on the health of a patient) bacteria. The field of probiotic-based pharmaceutical therapy is based on such a paradigm. One of the shortcomings of such an approach is the difficulty in targeting just the bad bacteria, which has already colonized in the intestines, to be eliminated. Therefore, a need still exists for a system and a method for affecting the intestinal microbial flora to benefit a patient.

### SUMMARY

Example 1 is a system for affecting intestinal microbial flora. The system comprises: an intestinal sleeve that is implanted in a patient and bypasses a section of the intestine; and probiotics, prebiotics or pharmacologic therapy used in combination with the intestinal sleeve.
Example 2 is the system of Example 1 further comprising an anchoring element, wherein the sleeve is anchored at a pyloric junction of a patient joining the stomach and the intestine by the anchoring element.
Example 3 is the system of Example 2 in which the anchoring element comprises two flanges, with one flange on an intestinal side and one flange on a stomach side.
Example 4 is the system of Example 2 in which the anchoring element is made from Nitinol.
Example 5 is the system of Example 2 in which the anchoring element further comprises a covering comprising a biocompatible polymer or fabric.
Example 6 is the system of Example 1 in which the sleeve is impregnated, coated or otherwise modified to include the probiotics, prebiotics or pharmacologic therapy.
Example 7 is a method of affecting intestinal microbial flora. The method comprises the steps of: implanting an intestinal sleeve in a patient that bypasses a section of the intestine; and administering probiotics, prebiotics or pharmacologic therapy to patient.
Example 8 is the method of Example 7 in which the sleeve is attached to an anchoring element that anchors the intestinal sleeve at a pyloric junction of the patient.
Example 9 is the method of Example 8 in which the anchoring element comprises two flanges, with one flange on an intestinal side and one flange on a stomach side.
Example 10 is the method of Example 8 in which the anchoring element further comprises a covering comprising a biocompatible polymer or fabric.
Example 11 is the method of Example 7 in which the sleeve is implanted in the patient for a finite period of time.
Example 12 is the method of Example 7 in which the probiotics, prebiotics or pharmacologic therapy is administered to the patient starting a few days before the sleeve is implanted.
Example 13 is the method of Example 7 in which the probiotics, prebiotics or pharmacologic therapy is administered to the patient when the sleeve is implanted.
Example 14 is the method of Example 7, further comprising the step of removing the sleeve from the patient.
Example 15 is the method of Example 14 in which the probiotics, prebiotics or pharmacologic therapy is administered to the patient starting a few days prior to removing the sleeve.
Example 16 is the method of Example 14 in which the probiotics, prebiotics or pharmacologic therapy is administered to the patient starting on the day the sleeve is removed.
Example 17 is the method of Example 7 in which the sleeve is implanted endoscopically.
Example 18 is the method of Example 14 in which the sleeve is removed endoscopically.
Example 19 is a method of affecting intestinal microbial flora in a patient. The method comprises the steps of: implanting a sleeve in the patient's intestine that bypasses a section of the intestine; and removing the sleeve after a period of time and after intestinal microbial flora has been affected.
Example 20 is the method of Example 19 in which the sleeve is impregnated, coated or otherwise modified to include probiotics, prebiotics or pharmacologic therapy.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows an enlarged view of the gastrointestinal anatomy at the junction between the stomach and the duodenum, including an anchoring element;

FIG. 2 shows another anchoring element having an alternate shape;

FIG. 3 shows an exemplary embodiment in which the anchoring element includes a cover;

FIG. 4 shows the anchoring element of FIG. 1 with a thin sleeve attached for carrying food from the stomach to the distal small intestine;

FIG. 5 shows the sleeve of FIG. 4 extending about 2 feet in length into the proximal jejunum;

FIG. 6 shows a simplified illustration of a patient's intestine that is suffering from a chronic disease as a result of an unfavorable balance between bad bacteria (circles) over good bacteria (stars);

FIG. 7 shows the patient's intestine (from FIG. 6) a few days after implantation of the sleeve described in FIG. 5;

FIG. 8 shows the patient's intestine after a period of approximately weeks after FIG. 7, and just prior to explantation of the sleeve;

FIG. 9 shows the patient's intestine after the sleeve has been explanted and probiotic therapy is initiated;

FIG. 10 shows an outcome of administering the probiotic therapy described in FIG. 9 for a period of several weeks; and

FIG. 11 shows a further continuation of the process described in FIG. 10 and a final outcome observed after the probiotic therapy is complete.

### DETAILED DESCRIPTION

The present invention involves an alternative or adjunct to probiotic therapy or other pharmacologic therapy to establish a new, more beneficial milieu of intestinal microbial flora. The result of good bacteria being more prevalent than bad bacteria in the intestine may resolve various ailments thought to be caused by the prevalence of bad bacteria over good. The present invention involves placing a sleeve within the intestine (i.e., gut) for a finite period of time to eradicate the colonization of the intestine by bacteria that play a role in metabolic disorders such as diabetes, obesity or general malaise (bad bacteria). In particular, an implant system may be used that can be placed by endoscopic means, anchored at the pyloric junction and connected to a sleeve component that extends into the jejunum. Placing the sleeve within a specific portion of the intestine can effectively starve the gut microbes of nutrients, reducing or altogether eliminating the bad gut microbes. The patient can then be administered probiotic therapy or other pharmacologic therapy while the implant is in place or after it has been removed to establish a healthier colony of bacteria in the intestine, thereby resolving various ailments. Also, alternatively, the sleeve itself could be used to deliver probiotic therapy while in place. This may include impregnating or coating the sleeve with probiotics or otherwise modifying the sleeve to carry such materials to a desired location within the anatomy.

The sleeve or implant system that may be used in the present invention may be of any suitable type. Exemplary embodiments of such an implant system or sleeve are depicted in the application, but other suitable devices or systems are also contemplated. Some particular exemplary devices or systems are included in a patent and a patent application assigned to the same assignee as the present invention, which are U.S. Pat. No. 8,211,186; and US Publication No. 2012/0253259, and are herein incorporated by reference.

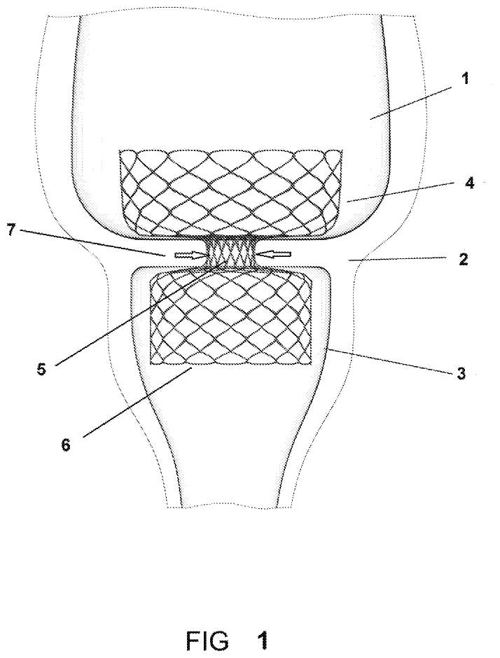

FIG. 1 shows an enlarged view of the gastrointestinal anatomy at the junction between the stomach and the duodenum, including the pyloric antrum 1, the pylorus 2, and the duodenal bulb 3. A soft, braided or laser-cut anchoring element 4 is placed at the pyloric junction (i.e., extending across the pylorus). The anchoring element may be made from Nitinol. The anchoring element 4 may be one component of an implant system that includes a sleeve that extends into the duodenum. As shown, the anchoring element 4 is shaped such that it does not exert radial forces on the stomach wall or the duodenal wall for anchoring when the pyloric junction (the pyloric canal and the sphincter) is in a relaxed state. It is retained within the pyloric junction due to its shape, which has an outer diameter larger than the maximum outer diameter of the pyloric orifice. The anchoring element 4 includes a proximal portion (i.e., the portion located in the pyloric antrum 1), a distal portion 6 (i.e., the portion located in the duodenal bulb 3, and a neck portion 5 adapted to extend through the pylorus 2. Additionally, during normal physiologic function of the pylorus 2, the tissue may exert a closing force 7 on the neck portion of the implant 5. In this case, the neck portion of the implant will compress and not inhibit normal function of the body. According to various embodiments, the proximal 4 and distal portion 6 of the implant are shaped such that each has an unconstrained diameter of between about 15 and about 35 millimeters, and the neck portion 5 has an unconstrained diameter of between about 5 and about 15 millimeters.

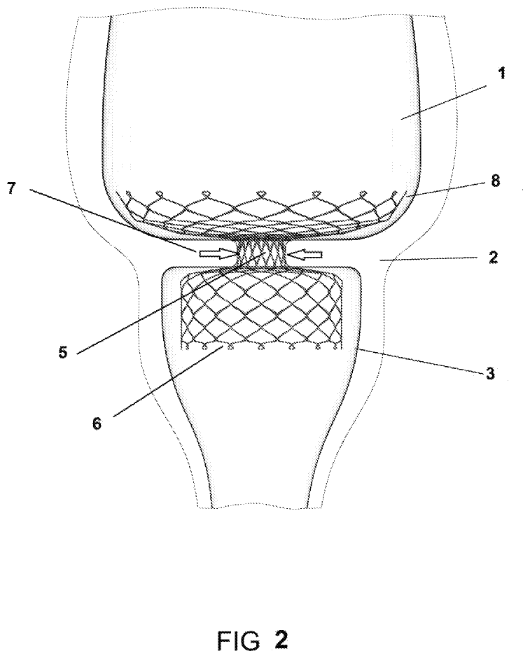

FIG. 2 shows another anchoring element 8 having an alternate shape. In this instance, the proximal portion of the anchoring element 8 (i.e., the portion located on the pyloric antrum side) is more disk-like and serves as a pronounced anchoring/retaining flange for the device. In some embodiments, the proximal portion of the anchoring element 8 has a maximum or unconstrained diameter slightly larger than an internal diameter of the pyloric antrum 1, such that the element exerts a slight radial force on the wall of the pyloric antrum 1.

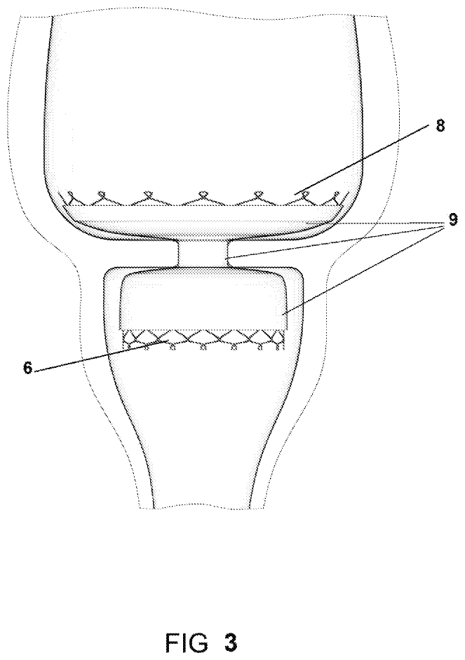

In order to minimize or prevent abrasive injury to tissue and tissue in-growth, and to provide for ease of replacement, exemplary embodiments of the anchoring elements shown in FIGS. 1 and 2 could be covered. FIG. 3 shows an exemplary embodiment in which the anchoring element 8 includes a cover 9. The material comprising the cover 9 could be a flexible woven fabric or nonwoven, extruded polymeric material used in synthetic medical grafts such as polyurethane, silicone, expanded polytetrafluoroethylene (ePTFE), or may be dip coated with such materials. Other suitable materials are also contemplated.

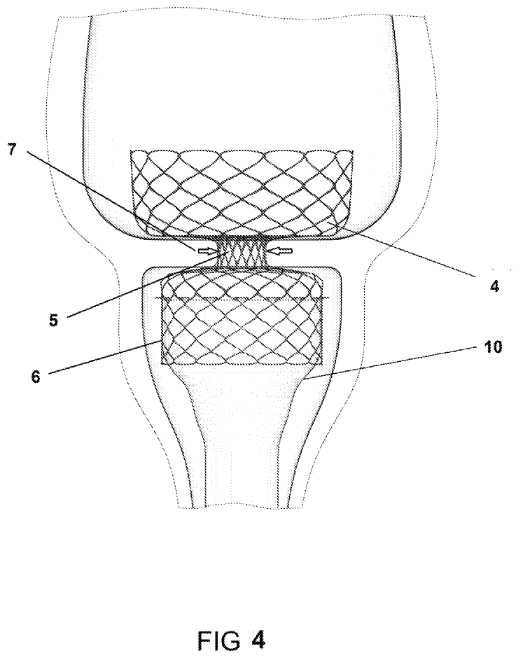

FIG. 4 shows the anchoring element 4 of FIG. 1 with a thin sleeve 10 attached for carrying food from the stomach to the distal small intestine. The sleeve 10 is preferably constructed of an impermeable material that is resistant to both stomach acid and alkaline intestinal secretions. The sleeve 10 may be made from biocompatible materials such as fluoropolymers (e.g., fluorinated ethylene propylene (FEP), perfluoroalkoxy (PFA) and ePTFE), silicone and polyurethanes, or soft metallic fabric structures made out of metals such as stainless steel. The wall thickness of the sleeve 10 is preferably in the range of 0.001 inches (0.03 mm) to 0.010 inches (0.25 mm) and the diameter of the sleeve is selected to match the diameter of the small intestine of the patient, usually between 20 to 30 mm.

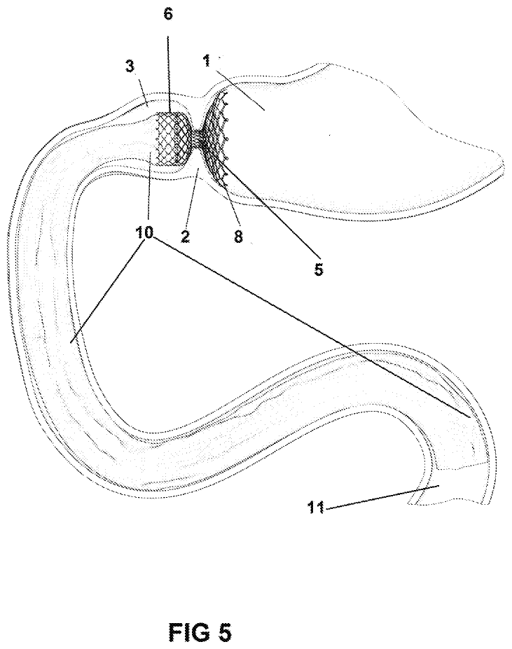

FIG. 5 shows the sleeve 10 of FIG. 4 extending about 2 feet (0.61 m) in length into the proximal jejunum 11. The sleeve 10 prevents food from contacting the wall of the proximal intestine and delivers it directly to the distal portion of the small intestine. The isolation of nutrients from the wall of the small intestine can starve the gut microbes or bacteria of essential nutrients in that region and can lead to amelioration of at least a certain percentage of such bacteria.

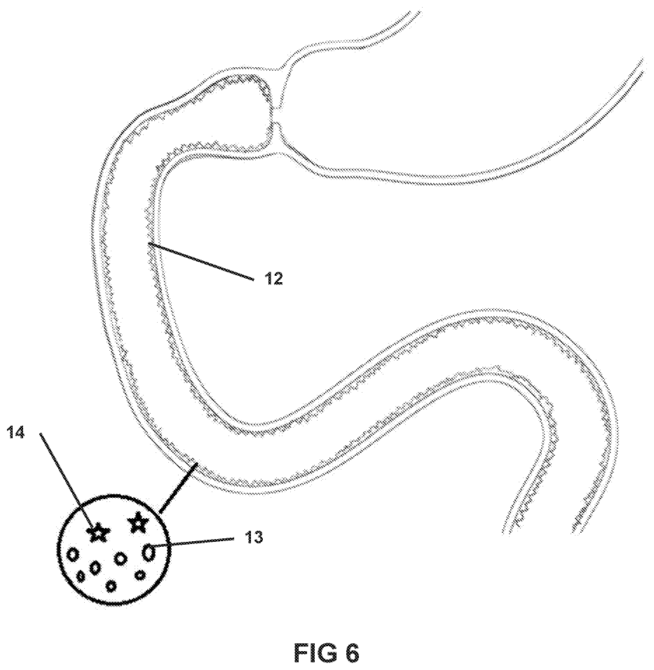

FIG. 6 shows a simplified illustration of a patient's intestine that is suffering from a chronic disease as a result of an unfavorable balance between bad bacteria (circles 13) over good bacteria (stars 14). The figure illustrates the problem that the therapeutic approach of this invention seeks to address. The illustration includes the same anatomy as shown in FIG. 5 prior to implantation of the sleeve 10.

In patients suffering from metabolic disorders such as type 2 diabetes and obesity, or autoimmune diseases such as rheumatoid arthritis and type 1 diabetes, the microbial flora lining the gut (or intestine) 12 is believed to be composed predominantly of bacteria that are not beneficial. For example, obese people are more likely to have a smaller proportion of Bacteroides (good bacteria) to Firmicutes (bad bacteria) within their guy. These bad bacteria 13 can effect nutrient absorption, signaling to the brain or participate in biochemical reactions that lead to chronic ailments. The type of bacterial colonies inhabiting a patient's gut might have origins in their diet, their environment and genetic factors. The present invention aims to change this microbial flora by initially eliminating a large portion of the bacteria by starving them of nutrition. Probiotic therapy may then be used to establish good microbial flora in the intestine.

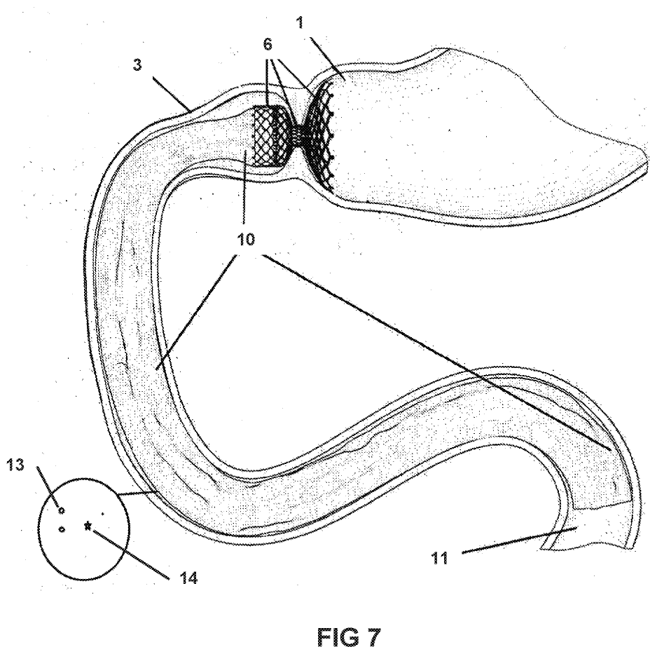

FIG. 7 shows the patient's intestine (from FIG. 6) a few days after implantation of the sleeve 10 described in FIG. 5. The effect of the sleeve 10 can already be seen as the composition of gut microbial flora has been modified. There is already a significant reduction in the number of bad bacteria 13 which may immediately result in remission of certain diseases. As an example, modification of the gut microbial flora after implantation of an intestinal sleeve in a patient with type 2 diabetes seems to cause normalization of glucose parameters within only a few days after the procedure. This modification also provides a foundation upon which the process of establishing new gut microbial flora can be started.

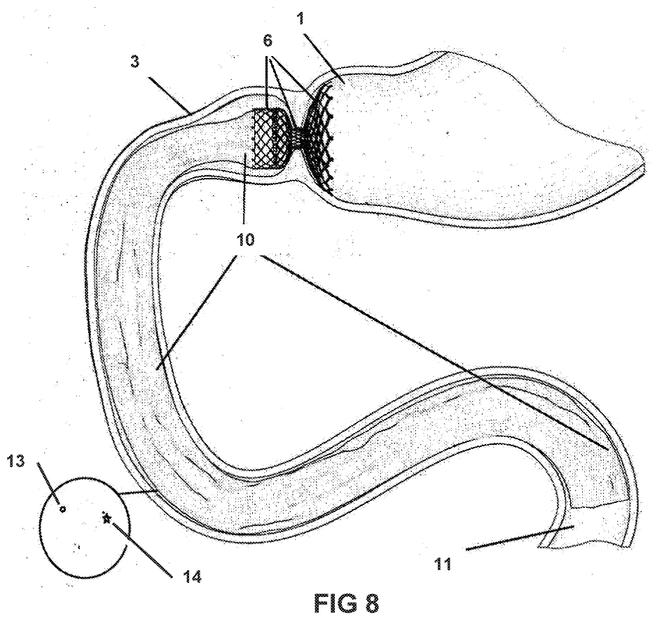

FIG. 8 shows the patient's intestine after a period of approximately weeks after FIG. 7, and just prior to explantation of the sleeve. The patient's anatomy has been further modified. The result is a significant reduction in the amount of bacteria in the gut. Here, the gut flora has been dramatically altered without the use of any antibiotics or pharmaceutical therapy, by simply preventing nutrients from reaching the gut microbes. This now provides a substrate for the repopulation of the gut with beneficial bacteria.

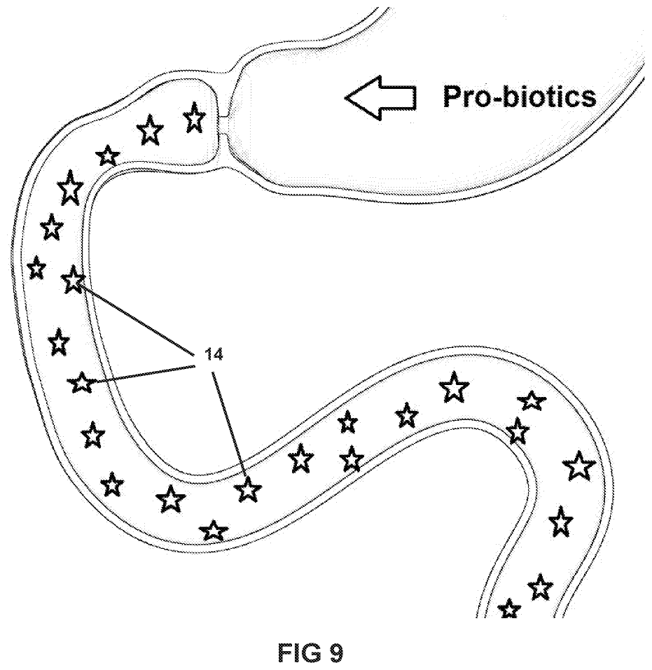

FIG. 9 illustrates the patient's intestine after the sleeve has been explanted and probiotic therapy is initiated. The goal of this step is to deliver beneficial bacteria 14 to the intestine in order to establish a healthier colony of microbes within the area modified by the sleeve. This may be accomplished as the patient consumes healthy meals following rules of a probiotic diet, through oral supplements or through specially prepared foods.

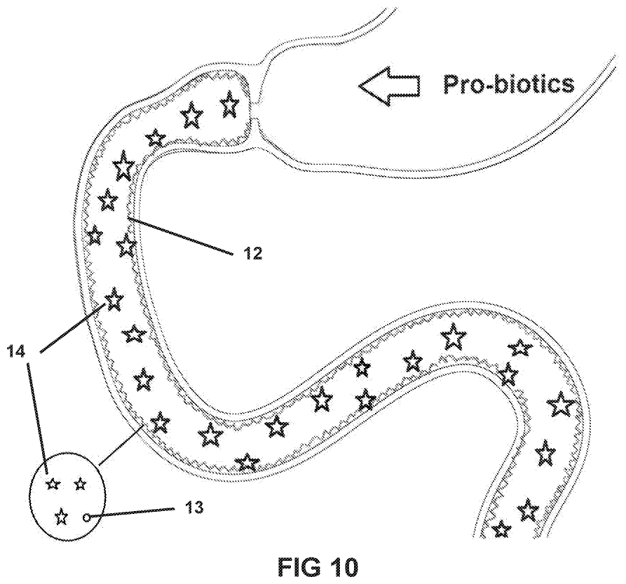

FIG. 10 illustrates the outcome of administering the probiotic therapy described in FIG. 9 for a period of several weeks. At this point, the therapy has resulted in the establishment of new gut microbial flora 12 with a beneficial balance of more good bacteria 14 than bad 13.

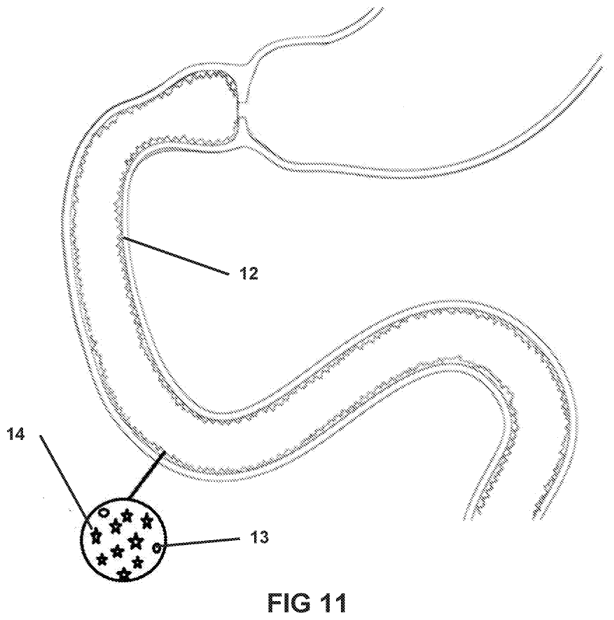

FIG. 11 illustrates a further continuation of the process described in FIG. 10 and the final outcome observed after the probiotic therapy is complete. Here the new healthy gut flora 12 has taken complete hold and is now composed of a greater percentage of good bacteria 14 than when the therapy was started. For the patient, the result will be remission of symptoms related to the original gut microbial imbalance, and possibly reversal of metabolic or auto-immune disorders such as obesity, diabetes or rheumatoid arthritis.

According to various embodiments of the invention, one or more of the systems described herein may be utilized to perform a therapeutic procedure including the following steps:
1. Implanting a thin intestinal sleeve that bypasses a specific section of the small intestine, in which the sleeve prevents nutrients from contacting the wall of the intestine and thereby helps to alter the microbial flora of that section of the intestine;
2. Removing the sleeve after a period of time. In various embodiments, the sleeve is removed after a period of three days. In other embodiments, the sleeve is removed anywhere from three days to three years after implantation; and
3. Administering probiotic or other pharmaceutical therapy to the patient to reestablish gut microbial flora that promotes health and helps reverse metabolic disorders, autoimmune diseases and other ailments, such therapy may be initiated before, right after implantation or upon explantation of the sleeve.

In 2006, researchers discovered that microbial populations in the gut are different between obese and lean people. The study, published in Nature (R. E. Ley, P. J. Turnbaugh, S. Klein, and J. I. Gordon, “Microbial ecology: human gut microbes associated with obesity,” Nature, vol. 444, no. 7122, pp. 1022-1023, 2006), further demonstrated that when obese people lost weight, their microflora reverted back to that observed in a lean person. More recently, additional studies have shown that microbial populations can be prospectively altered to achieve a desired change in the gut microflora. For example, a recent study published in The Journal of Biological Chemistry (Yadav, H et. al, Beneficial Metabolic Effects of a Probiotic via Butyrate-induced GLP-1 Hormone Secretion. J. Biol. Chem. 2013 288:25088-25097) showed that in animals, providing a probiotic dietary supplement containing a combination of eight bacterial strains;Bifidobacterium breve,Bifidobacterium longum,Bifidobacterium infantis,Lactobacillus acidophilus,Lactobacillus plantarum, Lactobacillus paracasei,Lactobacillus bulgaricus, andStreptococcus thermophiles; resulted in suppressed weight gain, lower blood glucose levels, and improved glucose and insulin tolerance compared to animals that did not receive the same therapy. The inventors recognized that the devices and methods disclosed herein could be effectively implemented to affect the microflora population in the intestines to capture the advantages set forth in these articles.

According to various embodiments of the present invention, any of the probiotics disclosed above may be used to complete the step of administering probiotics.

Other embodiments of the invention include the use of prebiotics in combination with an intestinal sleeve. Prebiotics, like probiotics, can play an important role in helping to colonize the gut with beneficial bacteria. Prebiotics are non-digestible food ingredients that function to stimulate the growth of bacteria in the digestive system. In the present invention, prebiotics could be administered in the same manner as described for probiotics, which is before, during and/or after sleeve implantation.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

1. A system for affecting intestinal microbial flora comprising:
an intestinal sleeve that is implanted in a patient and bypasses a section of the intestine; and
probiotics, prebiotics or pharmacologic therapy used in combination with the intestinal sleeve.

2. The system of claim 1, further comprising an anchoring element, wherein the sleeve is anchored at a pyloric junction of a patient joining the stomach and the intestine by the anchoring element.

3. The system of claim 2, wherein the anchoring element comprises two flanges, with one flange on an intestinal side and one flange on a stomach side.

4. The system of claim 2, wherein the anchoring element is made from Nitinol.

5. The system of claim 2, wherein the anchoring element further comprises a covering comprising a biocompatible polymer or fabric.

6. The system of claim 1, wherein the sleeve is impregnated, coated or otherwise modified to include the probiotics, prebiotics or pharmacologic therapy.

7. A method of affecting intestinal microbial flora comprising the steps of:
implanting an intestinal sleeve in a patient that bypasses a section of the intestine; and
administering probiotics, prebiotics or pharmacologic therapy to patient.

8. The method of claim 7, wherein the sleeve is attached to an anchoring element that anchors the intestinal sleeve at a pyloric junction of the patient.

9. The method of claim 8, wherein the anchoring element comprises two flanges, with one flange on an intestinal side and one flange on a stomach side.

10. The method of claim 8, wherein the anchoring element further comprises a covering comprising a biocompatible polymer or fabric.

11. The method of claim 7, wherein the sleeve is implanted in the patient for a finite period of time.

12. The method of claim 7, wherein the probiotics, prebiotics or pharmacologic therapy is administered to the patient starting a few days before the sleeve is implanted.

13. The method of claim 7, wherein the probiotics, prebiotics or pharmacologic therapy is administered to the patient when the sleeve is implanted.

14. The method of claim 7, further comprising the step of removing the sleeve from the patient.

15. The method of claim 14, wherein the probiotics, prebiotics or pharmacologic therapy is administered to the patient starting a few days prior to removing the sleeve.

16. The method of claim 14, wherein the probiotics, prebiotics or pharmacologic therapy is administered to the patient starting on the day the sleeve is removed.

17. The method of claim 7, wherein the sleeve is implanted endoscopically.

18. The method of claim 14, wherein the sleeve is removed endoscopically.

19. A method of affecting intestinal microbial flora in a patient comprising the steps of:
implanting a sleeve in the patient's intestine that bypasses a section of the intestine; and
removing the sleeve after a period of time and after intestinal microbial flora has been affected.

20. The method of claim 19, wherein the sleeve is impregnated, coated or otherwise modified to include probiotics, prebiotics or pharmacologic therapy.
